(19) United States
(12) Patent Application Publication
(10) Pub. No.: US 2004/0238379 A1
(43) Pub. Date: Dec. 2, 2004

(54) Nucleic acid field effect transistor

(76) Inventors: Stuart Lindsay, Phoenix, AZ (US); Trevor Thornton, Fountain Hills, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/486,035

(22) PCT Filed: Aug. 7, 2002
(86) PCT No.: PCT/US02/25019

Related U.S. Application Data
(60) Provisional application No. 60/310,992, filed on Aug. 8, 2001.

(51) Int. Cl. G01N 1/00; H01L 29/76; C12M 1/00; C12Q 1/00

(52) U.S. Cl. 205/792; 204/403.01; 257/213

(57) ABSTRACT

A method for electronically detecting hybridization of a probe nucleic acid and a target nucleic acid is disclosed. The probe nucleic acid (130) is attached to an open semiconductor channel (110) in a back-gated field effect transistor (120). A target nucleic acid is provided on the semiconductor channel, and electrical charateristics, such as the drain to source current, are monitored for changes indicating that hybridization has occured.

### CROSS-REFERENCE TO RELATED APPLICATIONS

This application claims the benefit of U.S. Provisional patent application Ser. No. 60/310,992 filed on Aug. 8, 2001 and entitled “DNA Field Effect Transistor” and incorporated by reference herein.

### STATEMENT REGARDING FEDERALLY SPONSORED RESEARCH

This invention was made with government support under Grant No. DMR-9632635 awarded by the National Science Foundation and Grant No. N00014-98-0594 awarded by the Office of Naval Research.

The present invention relates to the detection of hybridization of nucleic acid and more particularly to electronic devices for detecting hybridization of nucleic acid.

### BACKGROUND OF THE INVENTION

The human genome project has accentuated the need for rapid identification of the expression of particular genes in particular cells or organisms. The most promising technology for parallel detection is based on so called “genechips” (see “Light-generated oligonucleotide arrays for rapid DNA sequence analysis.” Pease, Solas, et al., Proc. Natl. Acad. Sci. (USA) 91:5022-5026, 1994; Fodor, Science 393, 1997). “Genechips” consist of arrays of spots of oligonucleotides attached to a solid (e.g., glass) substrate. Photo-deprotection and optical lithography permit many thousands of spots, each corresponding to a unique DNA sequence, to be “printed” onto a square-centimeter sized chip by the use of one mask for each base at each step of polymerization, so that an enormous number of sequences may be printed in just a few steps.

The genechip is usually incubated with fluorescently labeled target DNA and then rinsed. Hybridization is detected by fluorescence at the sites where target DNA (and its associated fluorescent tag) has bound. This detection scheme therefore relies on an intermediate step in which the target is combined with one or more fluorescent labels. For example, gene expression might be monitored by collecting the expressed mRNA and translating it to cDNA which is made from a labeled primer. After hybridization, the chip is illuminated with light that excites the fluorescent molecules and the location of the fluorescent spots is determined by confocal microscopy. Automated systems for doing this readout step are commercially available from Molecular Dynamics and Hewlett-Packard. They utilize automated image analysis of the illuminated, hybridized arrays to generate a map of the location of the hybridized DNA, and thus identify the target DNA. This approach is indirect. The optical readout step must be followed by image analysis and processing before the target DNA is identified, greatly complicating the readout process. Furthermore, the present approach requires labeling of target DNA.

It would be desirable to use electronic means to detect hybridization of target DNA with probe DNA, making the whole process capable of direct interfacing to a computer. In principle, this is a simple task, because the linear charge density associated with double stranded DNA is twice that of single stranded DNA. Even in the presence of screening counter ions, the change between single and double stranded DNA produces a significant time-averaged difference in local charge density. Near a depleted semiconductor surface, this change in charge density (or, correspondingly, surface potential) causes changes in a depletion layer near the semiconductor surface. This effect is exploited in a scanning probe potentiometer designed to locate regions of local change in charge density, such as tethered, hybridized DNA (Manalis, Minne, et al., Proc. Natl. Acad. Sci. (USA) 91:5022-5026, 1999). In the device described by Manalis, et al. (Manalis, Minne, et al., supra, 1999) photo-current from a small depleted region at the apex of a scanning probe is detected. Changes in charge density near the apex of the probe signal variations in charge. The device is designed to be scanned over a surface to which molecules are tethered, detecting hybridization of DNA, for example, as a local change in charge density.

In principle, the same approach could be used with a field effect transistor (FET), if the conducting channel could be exposed so that oligonucleotides could be attached, and changes in charge density detected as hybridization is carried out with target molecules. However, conventional FETs have gate electrodes covering the conducting channel. These not only obscure the channel, but they also require connections to be bought into the region of the device above the channel, making it incompatible with exposure of the channel to solutions.

The need for such a device goes beyond DNA hybridization. Any interaction that changed the charge associated with a biopolymer could be detected by such a device. Examples would be changes in oxidation state of a redox protein or binding by one polypeptide to another where there is a net change of charge.

Accordingly, it is an object of the present invention to provide a device for direct, electronic detection of biopolymer binding, such as DNA hybridization, compatible with the solution chemistry required for carrying out the binding. It is another object to eliminate the need of labeling of either the probe or target DNA. It is another object to construct a field effect transistor compatible with exposure to solutions both for attachment of DNA and for subsequent detection of hybridization.

### SUMMARY OF THE INVENTION

The present invention is a field effect transistor (FET) formed from a silicon-on-insulator layer on top of a semiconducting substrate. The silicon-on-insulator layer is separated from the substrate by a buried oxide layer. Drain and source electrodes are attached to the top silicon-on-insulator layer, which forms the conducting channel of the FET. An electrode is attached to the substrate, so that the substrate can be used as a back-gate to control the conductivity of the silicon-on-insulator channel. The top silicon-on-insulator layer is protected by an oxide layer, into which windows are etched to expose the surface of the silicon-on-insulator layer. When this surface is exposed to air a thin native oxide layer is formed. DNA oligomers or other nucleic acid biopolymers are attached to this thin native oxide layer in the window within the thicker protective oxide layer. Hybridization of the nucleic acid biopolymer is detected from the consequent shift in threshold voltage, or a shift in current at a given back-gate (V_{bg}) and drain (V_{ds}) bias. Hybridization is detected from the consequent shift in threshold voltage, or a shift in current at a given back-gate to-source bias (V_{bg}) and source-to drain bias (V_{ds}).

These and other aspects of the invention will become apparent from the following description. In the description, reference is made to the accompanying drawings which form a part hereof, and in which there is shown a preferred embodiment of the invention. Such embodiment does not necessarily represent the full scope of the invention and reference is made therefore, to the claims herein for interpreting the scope of the invention.

### BRIEF DESCRIPTION OF THE DRAWINGS

FIG. 1 is a schematic layout of the back-gated FET constructed in accordance with the present invention.

FIG. 2 is a schematic layout of a back-gated FET with source and drain connections in place and a protective layer and window opening above the channel.

FIG. 3 is a schematic layout of the back-gated FET with biomolecules attached to the native oxide layer above the channel.

FIG. 4 illustrates one scheme for covalent attachment of DNA to a native silicon oxide.

FIG. 5 is a chart illustrating current vs. gate-source bias for a back-gated FET with, and without an organic monolayer attached.

FIG. 6 is a schematic illustrating control elements used to correct for systematic changes in electrical output characteristics of the FET due to factors other than molecular binding.

FIG. 7 is a chart illustrating the source to drain current in a FET constructed in accordance with FIG. 3 when each of a non-hybridizing and a hybridizing DNA are applied.

### DESCRIPTION OF THE PREFERRED EMBODIMENTS

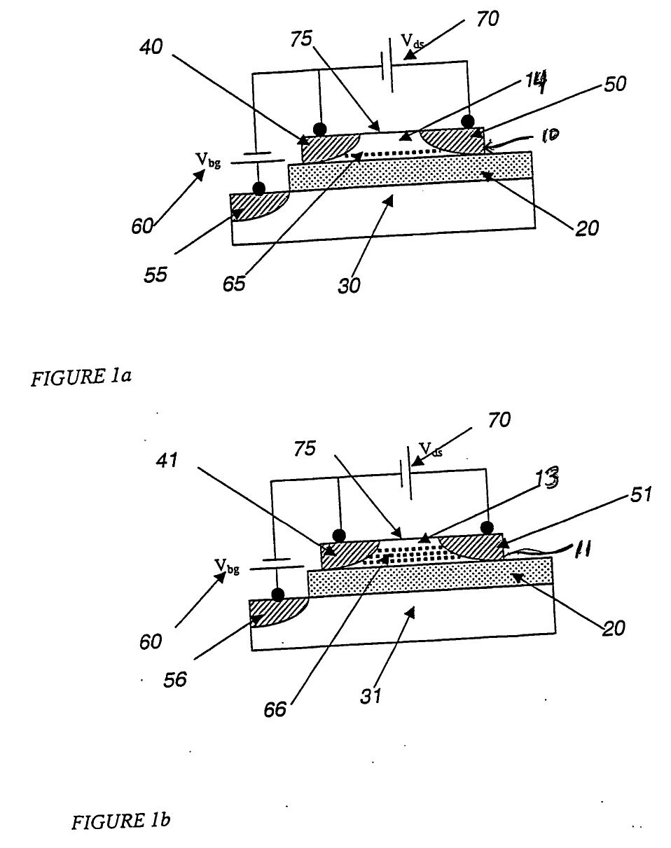

The current invention in its preferred embodiment is based on a back-gated field effect transistor (FET), shown schematically in FIG. 1. Basically, the back-gated FET comprises a semiconductor layer provided on an oxide insulating layer which is, in turn, provided on a conductive gate. The gate is therefore located on the back of the FET, as opposed to, for example, a MOSFET in which the gate is on top. The open semiconductor layer allows charges in a fluid placed on or in the semiconductor to interact with the semiconductor, as described below.

Referring still to FIG. 1, the FET as shown is built on a silicon on insulator (SOI) wafer available commercially from Ibis Corporation of Danvers, Mass. and is manufactured using a separation by implanted oxygen (SIMOX) process. Other sources and arrangements for the manufacture of silicon-on-insulator wafers are readily apparent to those skilled in the art, including wafer bonding and etch back as well as the SmartCut™ process. The FET consists of a layer of silicon 10 on top of a buried oxide (BOX) layer 20 that is, in turn, located on a silicon wafer 30 that serves as the substrate. The intrinsic surface layer of silicon 10 is typically 0.03 to 1 microns in thickness and the BOX layer 20 is typically 0.1 to 1 microns in thickness. Individual devices are isolated from each other by etching through the surface silicon layer 10, down to the BOX layer 10. The unetched areas of the surface silicon layer 10 are used to form the active regions of the device. The etching can be performed by wet chemical etching or reactive ion etching, as is well known in the art. Alternatively, the devices can be isolated using a well-known process called local oxidation of silicon (LOCOS). During LOCOS, the regions of the surface silicon layer 10 that are not required for the active regions are oxidized and the silicon in these regions is converted to insulating SiO₂.

In a preferred embodiment of a field effect transistor device constructed in accordance with the present invention, an n-channel inversion layer 65 is used to carry current between n-type source 40 and n-type drain 50 contacts as is shown in FIG. 1a. For this configuration, both the surface silicon layer 10 and the silicon substrate 30 are doped p-type, with typical doping concentrations in the range 10¹² to 10¹⁹ cm⁻³. Source 40 and drain 50 contacts are heavily doped n-type (e.g. with donor concentrations N_{D}˜10¹⁹-10²¹ cm⁻³) using ion implantation of, for example, phosphorus or arsenic, as is well known in the art. After implantation, conventional annealing or rapid thermal annealing at a temperature in the range 800-1000° C. is used to activate the implant and diffuse the contacts to such a depth that they reach the BOX layer 20. A p-type substrate or gate contact 55 is required to apply a back-gate voltage 60 to the substrate 30. The substrate contact 55 is readily made by first etching through the BOX layer 20 down to the substrate 30. The etch step is then followed by ion implantation of boron and rapid thermal annealing or conventional annealing to activate the dopants and form a heavily doped p-type region 55 (e.g. with boron concentration N_{A}˜10¹⁹-10²¹ cm³), as is well known in the art.

In the absence of an applied bias, this device is intrinsically non-conductive because of the lack of an inversion layer in the silicon layer 10 in the channel 14 between the source 40 and drain 50 connections. If, however, a bias voltage 60 (V_{bg}) is applied between source 40 and the substrate or gate contact 55 such that the substrate contact 55 is biased positive with respect to the source 40, minority electrons are attracted to the interface between the BOX layer 20 and the silicon layer 10, resulting in the electron inversion layer shown schematically by the dashed line 65 in FIG. 1. Thus, current will flow between the n⁺ source 40 and drain 50 connections when a bias voltage 70 is applied between them. Although the electron inversion layer 65 is formed next to the BOX layer 20 (as opposed to on the surface of the channel as in a normal FET) it is still extremely sensitive to charges placed on the upper surface 75 of the silicon layer 10.

It will be recognized by those skilled in the art that the same result may be achieved by replacing the electron inversion layer 65 with a hole inversion layer. For the case of a hole inversion layer an n-type SOI wafer would be used (with a typical doping concentration in the range 10¹²-10¹⁹ cm⁻³), along with heavily p-type doped source 40 and drain 50 contacts (with concentrations in the range of 10¹⁹-10²¹ cm⁻³). The back-gate voltage 60 would now be negative with respect to the source 40 contact.

Referring now to FIG. 1b, in another embodiment of the device current is carried between the source 41 and drain 51 contacts via majority carriers and it is therefore not necessary to induce a minority carrier inversion layer. For the case of current flow due to majority electrons, an SOI wafer with an n-type silicon-on-insulator layer 11 (N_{D}˜10¹²-10¹⁹ cm⁻³) would be used and separated from an n-type silicon substrate 31 (N_{D}˜10¹²-10¹⁹ cm⁻³) by a buried oxide layer 20. The source 41, drain 51 and substrate or gate 56 contacts for this case would now be heavily doped n-type with a donor concentration of, for example, (N_{D}˜10¹⁹-10²¹ cm⁻³).

When a bias voltage V_{ds} is applied to the drain 51, current flows in the silicon channel 13 and is not necessarily confined to the interface between the channel 13 and the BOX layer 20 as indicated by the multiple dashed lines 66. The current flowing in the channel 13 can be reduced (increased) by applying a back-gate bias voltage 60 to substrate contact 56 such that V_{bg} 60 is less than (greater than) zero. A negative back-gate voltage 10 reduces the electron concentration in the channel 13 and the current flowing between source 41 and drain 51 can be decreased to zero. Similarly, the current flowing in channel 13 can be increased by applying a back-gate bias voltage 60 which is greater than zero.

It will be recognized by those skilled in the art that the same result can be achieved in a majority carrier FET in which the current is carried by holes. In this configuration both the silicon channel 13 and the silicon substrate 31 would be p-type and doped in the range 10¹² to 10¹⁹ cm⁻³ and the source 41, drain 51 and substrate 56 contacts would be heavily doped p-type (e.g. with an acceptor concentration N_{A}˜10¹⁹-10²¹ cm⁻³).

Although the FET devices have been described above as constructed using a SIMOX wafer, other methods of forming the silicon-on-insulator channel will be apparent to those of skill in the art. For example, a poly-crystalline silicon (poly-Si) or amorphous silicon (α-Si) layer can also be used. In this embodiment a conventional silicon wafer is first oxidized to form a silicon dioxide (SiO₂) layer of thickness ˜0.05 to 2 μm on the surface. After growth of the SiO₂, chemical vapor deposition is used to deposit the poly-Si or α-Si to form a channel of thickness in the range 0.03 to 1 μm. The processing of the wafer to add the source, drain and back-gate contact electrodes would then proceed as described before. Again, a person skilled in the art will recognize that the poly-Si/α-Si versions of the device could be configured in such a way that the current in the silicon-on-insulator channel flows through an electron (or hole) inversion layer or an electron (or hole) accumulation/depletion layer. Although the electron (or hole) mobility in the poly-Si/α-Si embodiments of the device would be substantially less than that in a single crystal SIMOX, or wafer-bonded or SmartCut™ SOI wafer, their electrical characteristics would be sufficiently similar to enable their use in the electronic detection of DNA hybridization.

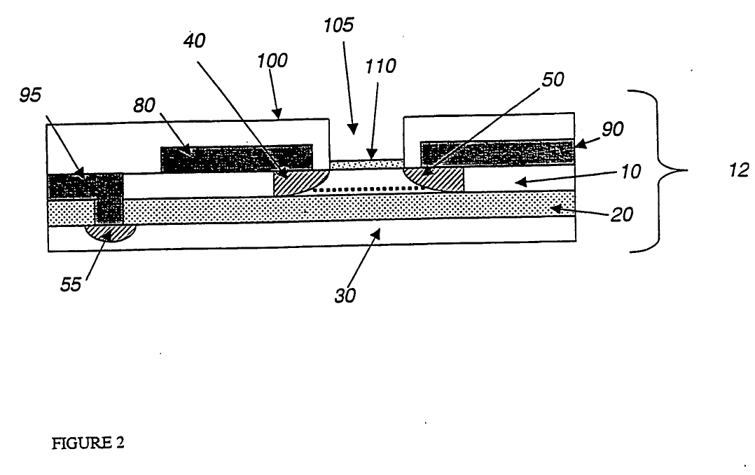

Referring now to FIG. 2, the device of FIG. 1a is shown encapsulated in a way that permits the upper surface 75 of the channel 14 to be exposed to solutions. Metallic connections 80, 90, 95 are made by deposition of, for example, aluminum, so as to contact the source 40, drain 50 and gate or substrate contacts 55, respectively. The connections 80, 90, 95 can be deposited by, for example, evaporation or sputter coating as is well known in the art. A passivating layer 100 of silicon dioxide or silicon nitride is applied to a thickness of between 50 and 1000 nm using standard deposition techniques such as chemical vapor deposition or spin-on-glasses. A window 105 is etched into the passivating layer 100 by standard lithographic procedures, arranged so as to expose the upper surface of the SOI 10 in the channel region between the source 40 and drain 50 diffusions. For example, one method of fabricating the window 105 is to use a patterned photoresist as a mask for a subsequent etch step using selective acid etches such as hydrofluoric acid, or by reactive ion etching, both of which are well known in the art. Ina preferred embodiment, an SU8 resist is used in order to provide a deep channel for fluids contained in the window, as described below. In the next step a thin oxide layer 110 is grown over the exposed region of SOI 10. One method to do this is to exploit the native oxide that grows naturally on a bare surface of silicon exposed to air at room temperature. Alternatively, a thermal oxide layer can be grown by heating the silicon to 800-1100° C. and exposing the surface to oxygen or steam. A typical thickness of this layer ranges from 2 nm to 100 nm. Electrical connections can now be made to the entire FET 120 consisting of source 80 and drain 50 and back-gate 95 in any hermetically-sealed package that has a widow exposing the oxide-coated channel 110.

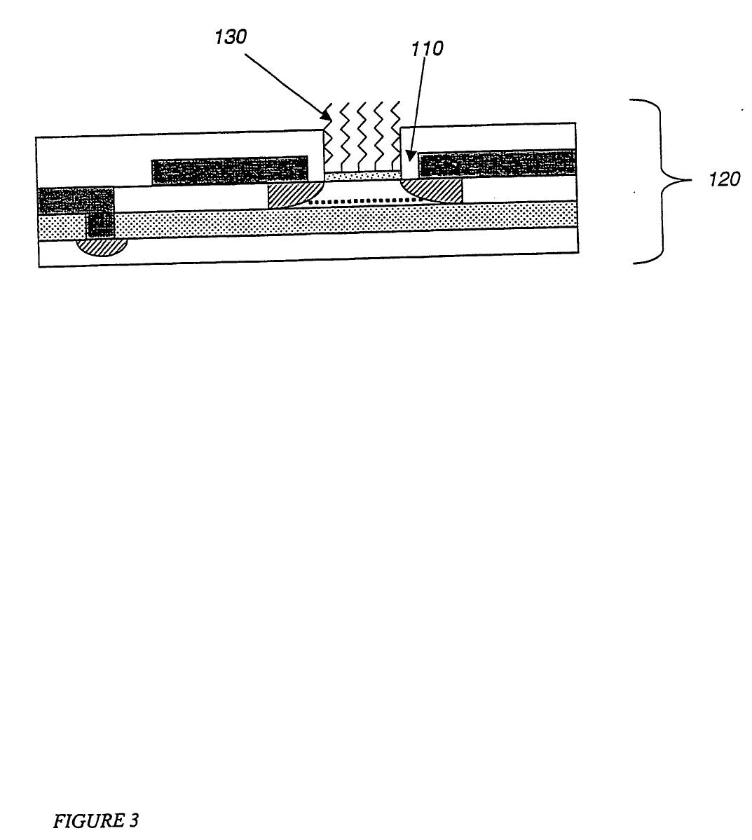

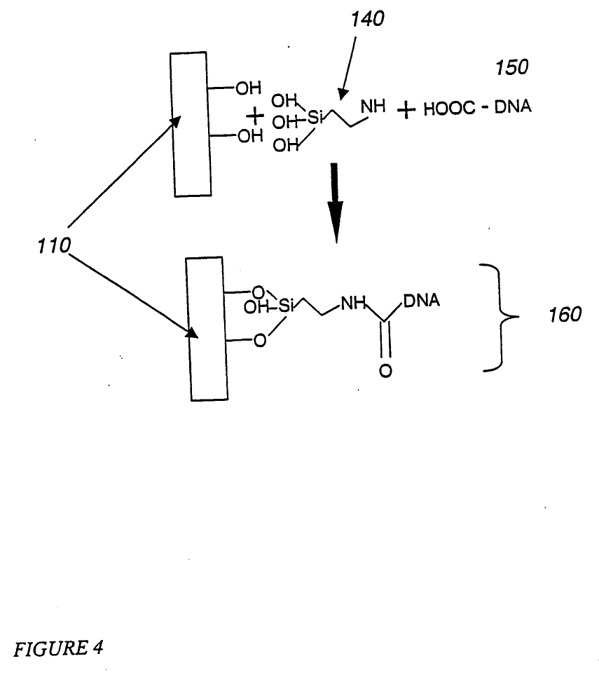

Referring to FIG. 3, biopolymers 130 are attached to the exposed oxide layer 110 using suitable chemical procedures, preferably by a covalent bond, although other weaker attachments can also be used. The attached biopolymer includes a probe for determining hybridization by a target solution, as described below. Preferred biopolymers include both synthetic and natural DNA and RNA. Changes in the charge density associated with changes in this biopolymer layer will alter the surface potential of the channel 10 between the source 40 and drain 50 diffusions, and so be detected as a change in the electrical properties of the FET. An example of one chemical probe attachment process is shown in FIG. 4. Here, a carboxylated DNA oligomer 150 is attached to the oxide layer 110 via a hydrolyzed silane 140 according to the procedure described by Zammatteo, et al. (Zammatteo, Jeanmart, et al., Analytical Biochemistry 280:143-150, 2000). The OH groups on the surface of the native silicon oxide layer 110 are naturally present. Silanizing agents such as 3′-amino-propyl tri (ethoxy silane) are readily available (from, e.g., Sigma Aldrich) and, on contact with water, or water vapor, hydrolyze to form the compound 140 shown in FIG. 4. The primary amine reacts with the carboxy group on the DNA to form a stable amide bond, and the hydroxyl groups on the silicon compound 140 react with hydroxyl groups on the surface oxide layer 110, forming the bound complex 160 shown in the lower part of FIG. 4. Carboxylated DNA oligomers are available from Midland Certified Reagent Company and are synthesized to any desired sequence starting with a carboxy dT.

There are many other approaches to covalent attachment of DNA to a silicon oxide surface. Examples are attachment of aminated DNA (Zammatteo, Jeanmart, et al, supra, 2000), phosphorylated DNA (Zammatteo, Jeanmart, et al., supra, 2000), thiolated DNA (Halliwell and Cass, Analytical Chemistry 73:2476-2483, 2001) and direct synthesis of oligomers on the glass surface (Pease, Solas et al., supra, 1994).

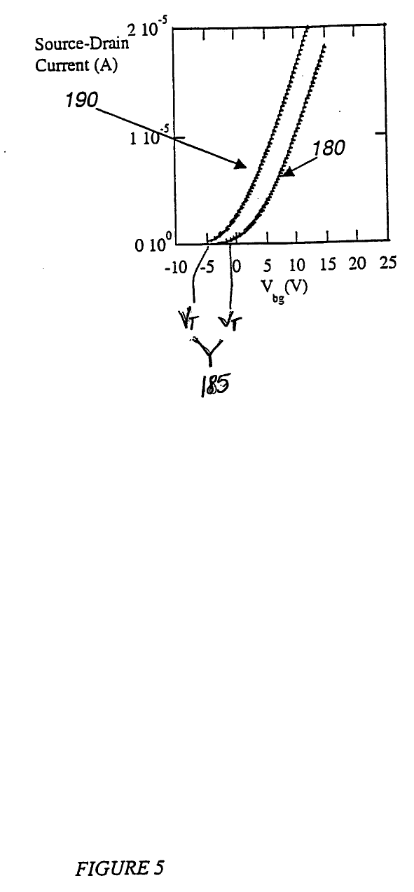

The presence of an organic monolayer on the surface of the channel 110 leads to large changes in the electrical properties of the electron inversion layer FET of FIG. 1a and FIG. 4 as illustrated in the graph of FIG. 5. Here, bias voltages are applied to drive the FET into the active region, and electrical characteristics of the FET are monitored to determine the change in electrical characteristics. The graph of FIG. 5 illustrates the source-drain current measured at a source-drain bias voltage 70 of 1.0V as a function of the source to back-gate bias voltage (V_{bg}) 60 for a bare oxide layer (curve 180) or an oxide layer with an organic monolayer attached (curve 190). The shift in threshold voltage, i.e. the applied back-gate bias voltage 60 between the source 40 and drain 50 required to cause measurable current to flow from the drain 50 to source 40 is about 4V in this case. Changes in the drain to source current flow can also be monitored as an indication of changes in the semiconductor channel. Even quite subtle rearrangements of the organic layer cause significant changes to the threshold voltage 185 of the FET, and these changes are used to detect, for example, hybridization of DNA. The voltage shifts depend on the specific chemistry used to bind the biopolymer probe 130 to the surface 110 and on the conditions used to achieve binding (or unbinding) with the probe 130, but a self calibrated device can compensate for conditions used to achieve binding as described below.

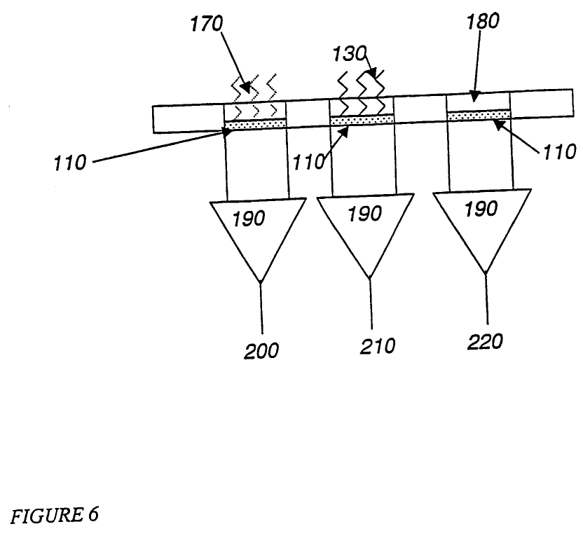

Alterations of electrical behavior caused by changes such as DNA hybridization are predictable and reproducible if well-controlled and clean conditions are used to carry out the reactions. This is not always possible, nor practically desirable. For this reason the FET preferably includes control elements as shown in FIG. 6. Here, a probe 130 comprising DNA is shown attached to the channel oxide 110 of one FET, and the channel current is monitored by a current to voltage converter 190, giving a voltage output 210 sensitive to the state of the probe DNA 130 when the FET is biased appropriately, i.e. providing a signal indicative of whether hybridization has occurred. The same wafer includes FET devices with blank channel oxides 180 and FET devices with channels functionalized with a non-hybridizing DNA sequence 170 selected not to hybridize with molecules in the solution being tested. The output of the device is based on differential measurements made between the probe device output 210 and the control outputs 220 and 200 as hybridization (or conversely, melting) reactions are carried out. Signals provided by the blank channel 180 normalize for environmental conditions such as salts present, concentrations of reagents, temperature, pH, and other factors which affect the characteristics of the transistors regardless of whether hybridization has occurred. Signals produced by the non-hybridizing DNA sequence channel 120 are used to normalize for effects owing to unspecific DNA-DNA interactions other than proper Watson-Crick base repairing. Each of the outputs 210, 220, and 230 can be provided to a computer or other device including a central processing unit programmed to normalize the output 210 based on the signals at outputs 220 and 230. Normalization can be provided, for example, using a look-up table, an algorithm, or using other methods apparent to those of skill in the art.

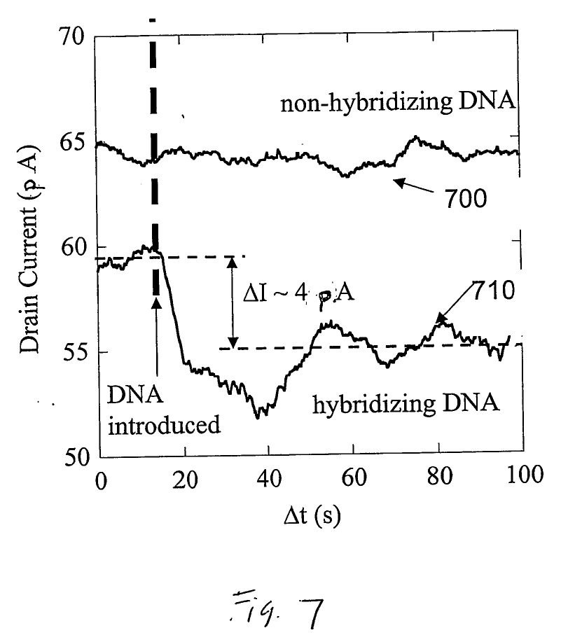

Referring now to FIG. 7, a chart illustrating the drain 50 to source 40 current as a function of time for a FET constructed in accordance with FIG. 2 is shown as each of a non-hybridizing target DNA and a hybridizing target DNA are applied to the surface 110 including probe 130, comprising an oligomer. To obtain these results, the open oxide window 105 of surface 110 in FIG. 2 was exposed to APTES as described above to produce the amine-functionalized surface as shown as 140 in FIG. 4. An improved approach (described in Facci P, Alliata D, Andolfi L. 2002. Formation and characterization of protein monolayers on oxygen-exposing surfaces by multiple-step self-chemisorption. Surf. Sci. 504:282-292) was used to attach a probe 130, an amine-modified oligomer as follows: the APTES modified window 105, in layer 110, was briefly exposed to a 1 mM solution of glutaraldehyde to place reactive aldehyde groups on the surface. These are, in turn, exposed to a solution of an amine modified oligomer, specifically:

| | |
|---|---|
| 5′ Amine-c6 spacer-gatccagtcggtaagcgtgc-3′ | (SEQ ID NO: 1) |

This is comprised of the following oligomer

gatccagtcggtaagcgtgc

with an amine attached via a 6-carbon alkane spacer. The probe sequences may be longer than SEQ ID NO: 1, preferably less than 1 MB, more preferably less than 1 KB and most preferably less than 100 bp. The amine reacts covalently with the gltuaraldehyde modified surface to tether the DNA as described above. The resulting device configuration is as shown in FIG. 3 with the oligomer tethered to the oxide window 110 as the probe DNA 130.

The operation of the FET is demonstrated by a plot of drain 50 to source 40 current versus time of FIG. 7. During the measurement an applied drain-source bias voltage 70 is kept constant at Vds=1 V and the backgate voltage 60 is grounded i.e. Vbg=0V. A non-hybridizing target sequence:

| | |
|---|---|
| 5′ agttagcatcactccacga 3′ | (SEQ ID NO: 2) |

was introduced to the FET device in a buffer maintained at 80° C. (having previously been exposed to the heated buffer with no added DNA). The heavy dashed curve marks the point at which the target DNA was added, and the current trace 700, shows no significant response to the non-hybridizing target DNA.

The measurement was then repeated with the addition of a hybridizing sequence:

| | |
|---|---|
| 5′ cacgcttaccgactggatc 3′ | (SEQ ID NO: 3) |

A preferable hybridizing sequence has no more than 10% mismatch within the hybridizing region. Almost immediately after the hybridizing target DNA is introduced into the photoresist opening or window 105 in the oxide layer 110 in FIG. 2, the drain to source current drops and stabilizes at an approximately constant value, about 4 pA lower than before the target DNA is introduced, as shown by the lower curve 710 of FIG. 7. Because the carriers in the test FET are electrons, the reduction in current is an expected consequence of the accumulation of extra negative charge on the oxide as the probe DNA 130 hybridizes with the target DNA.

To create a genechip, a plurality of FETS as described above are constructed to include a different sequence on each FET, preferably including at least some FETS that include a “control” built with a non-hybridizing DNA as described above. When target DNA is injected, a computer identifies the sequence based on the electrical charges of the FET as described above, and, by analyzing the results can also provide a measure of the relative concentrations of the DNA or nucleic acid. Therefore, total gene expression and relative level of gene expression can both be mapped.

It should be understood that the methods and apparatuses described above are only exemplary and do not limit the scope of the invention, and that various modifications could be made by those skilled in the art that would fall under the scope of the invention. To apprise the public of the scope of this invention, the following claims are made:

1. A field effect transistor, comprising
a semiconductor channel contacted by source and drain connections;
a buried oxide layer coupled to a first side of the semiconductor channel and a surface oxide layer coupled to an opposing side of the semiconductor channel;
a conducting layer coupled to a side of the buried oxide layer opposed to the semiconductor channel, the conducting layer providing a gate connection; and
a layer of nucleic acid biopolymeric material covalently bonded to the surface oxide layer.

2. The device of claim 1 wherein the semiconductor channel is a silicon.

3. The device of claim 1 wherein the semiconductor channel is a doped silicon.

4. cancelled

5. The device of claim 1 wherein the conducting layer is a doped silicon.

6. The device of claim 1 wherein the biopolymer is DNA.

7-8. cancelled

9. A device for detecting the attachment of a target nucleic acid biopolymer to a surface comprising:
a back-gated field effect transistor having a semiconductor channel in a top layer coupled to a buried oxide layer; and
a layer of probe nucleic acid biopolymeric material attached to the semiconductor channel, the probe biopolymeric material chosen to covalently bind to said target nucleic acid biopolymer, wherein an electrical characteristic of the field effect transistor changes as the target biopolymer binds to the probe biopolymeric material.

10. The device of claim 9 where the channel is silicon.

11. The device of claim 9 where the channel is doped silicon.

12. The device of claim 9 where the biopolymer is DNA.

13. The device of claim 9 further comprising a native oxide layer covering the channel.

14. The device of claim 13 in which the biopolymer is attached to the native oxide layer.

15. The device of claim 9, further comprising at least one back-gated field effect transistor that does not include a layer of probe biopolymeric material.

16. The device of claim 9, further comprising at least one back-gated field effect transistor including a layer of non-hybridizing DNA attached to the semiconductor channel.

17. The device of claim 12, further comprising at least one back-gated field effect transistor wherein the semiconductor channel is blank and at least one back-gated field effect transistor wherein the semiconductor channel includes a layer of non-hybridizing DNA.

18. The device of claim 17, wherein each of the back-gated field effect transistors is electrically coupled to a comparator, and an output signal of the comparators coupled to the blank back-gated field effect transistor and an output signal of the comparator coupled to the non-hybridizing field effect transistor ate used to normalize an output signal of the comparator coupled to the back-gated field effect transistor including the DNA.

19. The device as defined in claim 18, wherein the outputs of the back-gated field effect transistor are coupled to a comparator.

20. A method for detecting the attachment of a target nucleic acid biopolymer to a surface comprising:
selecting a probe biopolymeric material to covalently bind to a selected target nucleic acid biopolymer;
attaching a layer of nucleic acid biopolymeric material to a native oxide layer provided on a semiconductor channel in a back-gated field effect transistors wherein the layer comprises the probe nucleic acid;
applying the target nucleic acid biopolymer to the back-gated field effect transistor;
applying a voltage bias to the field effect transistor to drive the field effect transistor into an active range; and
monitoring an electrical characteristic of the field effect transistor for a charge indicating that the probe nucleic acid biopolymeric material and the target nucleic acid biopolymeric material have hybridized.

21. The method of claim 20 wherein the electrical characteristic is a threshold voltage.

22. The method of claim 20 where the electrical characteristic is a change in the drain to source current produced at a selected applied back-gate voltage and drain to source voltage.

23. The method of claim 20 where the nucleic acid biopolymer is DNA.

24. cancelled

25. The method of claim 24 further comprising the step of attaching the nucleic acid biopolymer to said native oxide layer.

26. The method as defined in claim 20, further comprising the steps of:
applying a voltage bias to a second back-gated field effect transistor;
monitoring the electrical characteristics of the second back-gated field effect transistor; and
normalizing the output of the back-gated field effect transistor for environmental conditions based on the monitored electrical characteristics.

27. The method as defined in claim 20, further comprising the steps of:
selecting the probe nucleic acid biopolymeric as a probe DNA;
selecting a non-hybridizing DNA that does not hybridize with the probe DNA material as a comparator to the DNA;
attaching a layer of the non-hybridizing DNA material to a semiconductor channel in a second back-gated field effect transistor;
applying a voltage bias to the second back-gated field effect transistor and using the output of the second back-gated field effect transistor to normalize the electrical characteristic of the back-gated field effect transistor for a DNA-DNA interaction other than Watson-Crick base repairing.
